(19) United States
(12) Patent Application Publication
(10) Pub. No.: US 2010/0069377 A1
(43) Pub. Date: Mar. 18, 2010

(54) TREATMENT OF FEMALE SEXUAL DYSFUNCTION BY COMPOUNDS THAT POSITIVELY MODULATE AMPA-TYPE GLUTAMATE RECEPTORS

(75) Inventors: Danielle Simmons, Palo Alto, CA (US); Gary Lynch, Irvine, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 12/532,399

(22) PCT Filed: Mar. 13, 2008
(86) PCT No.: PCT/US2008/056793

§ 371 Date: Nov. 18, 2009

Related U.S. Application Data
(60) Provisional application No. 60/896,617, filed on Mar. 23, 2007.

(51) Int. Cl. A61K 31/5365; A61K 31/498; A61P 15/00

(52) U.S. Cl. 514/229.5; 514/249

(57) ABSTRACT

The present invention provides methods of increasing sexual arousal and performance behaviors in female mammals by administration of a therapeutically acceptable amount of a positive modulator of an AMPA-type glutamate receptor.

### CROSS-REFERENCE TO RELATED APPLICATIONS

This application claims the benefit of U.S. Provisional Application No. 60/896,617, filed on Mar. 23, 2007, the entire disclosure of which is hereby incorporated herein by reference in its entirety for all purposes.

### FIELD OF THE INVENTION

The present invention is directed to the treatment of low female libido and sexual performance by administration of compounds that positively modulate AMPA-type glutamate receptors.

### BACKGROUND OF THE INVENTION

Female sexual dysfunction (FSD), including low sexual desire and arousal disorders, is the most common sexual problem among women, reported by 10 to 51 percent of women surveyed in various countries (Basson, New England J Med (2006) 354:1497-1506). FSD includes disorders of libido, arousal, orgasm, and sexual pain that lead to personal distress or interpersonal difficulties (Pauls, et al., Obstetrical & Gynecological Survey (2005) 60:196-205). The frequency of FSD increases with age, and menopause negatively affects sexual motivation or “libido”. These deficits are due to decreasing levels of estrogen (E) and progesterone (P) (for review, see for example, West et al., Annual Review of Sex Research (2004) 15: 40-172). Pharmacological treatment options for FSD associated with menopause include systemic hormone therapy including estrogen, E+P, E+testosterone, or tibolone, and have been shown to positively impact the condition (West et al., 2004, supra). However, use of hormone therapy for menopause has been shown to increase the risk of breast cancer and cause other negative side-effects (Rosenberg, Breast Cancer Research (2006) 8:R11 (doi:10.1186/bcr1378)). Therefore, other treatments that do not have these side-effects are desirable.

The gonadal hormones E and P act on the central nervous system to control female sexual behavior. Female sexual motivation and performance depends on ovarian release of E which primes brain cells for the later release of P by increasing P receptors (for review, see Math, J Mol Endocrinol. (2003) 30:127-37). E and P act synergistically in the brain to regulate sexual behavior especially through their actions in the ventrolateral part of the ventromedial hypothalamus (VMHv1) and medial preoptic area (MPOA) of the hypothalamus. Primarily, the MPOA is involved in the motivational aspects of female sexual behavior while the VMHv1 is involved with performance.

Both E and P can influence female sexual behavior by regulating the production or signaling of neurotransmitters. One excitatory neurotransmitter that influences sexual behavior is glutamate. Both E and P receptors are found on the same cells as those that contain glutamate receptors, N-methyl-D-aspartate (NMDA) and amino-3-hydroxy-5-methyl-4-isoxazolepropionic acid (AMPA), in the VMH and MPOA (Diano et al., Endocrinology (1997)138:778; Calizo et al., J. Neurosci. (2000) 20:1589). This finding suggests that E and/or P may directly affect glutamate transmission in brain areas that control female sexual behavior. In fact, glutamate inhibits sexual motivation and performance in female rats when injected into the VMHv1 (Georgescu and Pfaus, Pharmacol Biochem Behav (2006) 83:322 and Georgescu and Pfaus, Pharmacol Biochem Behav (2006) 83:333). This was true whether it acted at either its AMPA or NMDA receptors (Georgescu and Pfaus, supra). Furthermore, infusion of selective glutamate receptor antagonists into the VMHv1 facilitated female sexual behavior (Georgescu and Pfaus, supra).

Although the above studies indicate that glutamate acting at AMPA receptors in the VMHv1 inhibits female sexual behavior, the effect of its actions at AMPA receptors in other brain areas that affect female sexual behavior is unknown. There remains a need for pharmacological treatments that enhance female sexual arousal and/or performance without undesirable side effects. The present invention addresses this and other needs.

### BRIEF SUMMARY OF THE INVENTION

In a first aspect, the present invention provides methods for alleviating or decreasing sexual dysfunction in a female mammal. In some embodiments, the methods comprise administering a therapeutically effective amount of a compound that positively modulates α-amino-3-hydroxy-5-methyl-isoxazole-4-propionic acid (“AMPA”)-type glutamate receptors in said subject, said modulation being sufficient to decrease the symptoms of sexual dysfunction in the female mammal, wherein the female mammal does not otherwise need a positive modulator of the AMPA receptor.

In some embodiments, the female mammal is human. In some embodiments, the female mammal is a domestic mammal, an agricultural mammal or a laboratory mammal. In some embodiment, the methods are carried out with the provision that the mammal not be a rodent.

In some embodiments, the decreasing sexual dysfunction comprises increasing sexual arousal. In some embodiments, the decreasing sexual dysfunction comprises increasing sexual performance.

In some embodiments, the female mammal has low serum estrogen levels. For example, the treated female mammal can have a total serum estradiol level of less than about 500 pmol/l, or total serum estrogen levels of less than about 50 ng/ml. In some embodiments, the female mammal has total serum follicle-stimulating hormone levels of less than about 2 IU/l or more than about 9 IU/l.

In some embodiments, the female mammal is perimenopausal, post-menopausal or ovariectomized. In some embodiments, the female mammal has been diagnosed with a sexual dysfunction disorder. In some embodiments, the female mammal has been diagnosed with a female sexual dysfunction condition selected from the group consisting of Hypoactive Sexual Desire Disorder (DSM IV #302.71), Sexual Aversion Disorder (DSM-IV #302.79), Female Sexual Arousal Disorder (DSM-IV #302.72), Female Orgasmic Disorder (DSM-IV #302.73), Dyspareunia (DSM IV #302.76) and/or Vaginismus (DSM-IV #306.51).

In some embodiments, the compound is administered orally. In some embodiments, the compound is administered parenterally.

In some embodiments, the compound is a low impact positive modulator of the AMPA-type glutamate receptor. In some embodiments, the compound is a high impact positive modulator of the AMPA-type glutamate receptor. In some embodiments, the compound is selected from the group consisting of CX516, CX614 and CX689.

### DEFINITIONS

Female “sexual dysfunction” generally denotes the inhibition of any one or more of the phases of sexual response (appetite, arousal, excitement, orgasm, resolution) described in the Diagnostic and Statistical Manual of Mental Disorders (DSM IV, 2000, American Psychiatric Association). “Sexual dysfunction” specifically encompasses decreased sexual desire (Hypoactive Sexual Desire Disorder, DSM-IV #302.71; Sexual Aversion Disorder, DSM-IV #302.79), decreased sexual arousal (Female Sexual Arousal Disorder, DSM-IV #302.72), the inability to experience orgasm (Female Orgasmic Disorder, DSM-IV #302.73), Sexual Pain Disorders (Dyspareunia, DSM-IV #302.76; Vaginismus, DSM-IV # 306.51), and sexual dysfunction due to general medical conditions. The DSM-IV definitions and text relating to sexual dysfunction are hereby incorporated by reference (see, chapter on Sexual and Gender Identity Disorders on pages 535-582). Female “sexual dysfunction” includes lack of sexual motivation (libido) or performance in a female mammal. Female sexual dysfunction can occur for any of a number of reasons. Sexual dysfunction may be psychogenic only, or psychogenic and biogenic, lifelong or acquired, and generalized or situational. For example, female sexual dysfunction can be caused by physical illness, medications to treat other indications, surgery (e.g., subsequent to a hysterectomy), psychological factors and/or menopause. Female sexual dysfunction can physically manifest as decreased libido, arousal, orgasm, and/or increased sexual pain and socially/psychologically manifest as personal distress and/or interpersonal difficulties.

The phrase “symptoms of sexual dysfunction” includes inhibition of any of the four phases of sexual response (appetite, excitement, orgasm, resolution) outlined in the DSM-IV. These specifically include lack of sexual desire (Hypoactive Sexual Desire Disorder, DSM-IV #302.71; Sexual Aversion Disorder, DSM-IV #302.79), decreased sexual arousal (Female Sexual Arousal Disorder, DSM-IV #302.72), the inability to experience orgasm (Female Orgasmic Disorder, DSM-IV #302.73), loss of libido, decreased sexual performance and/or inability to perform adequately (i.e. insufficient vaginal lubrication and/or failure to orgasm, e.g., Sexual Pain Disorders (Dyspareunia, DSM-IV #302.76; Vaginismus, DSM-IV # 306.51).

“Age-related sexual dysfunctions” are sexual dysfunctions that are manifested in aging subjects and that often worsen with increasing age. They are common to both human and animal species (Reviewed in, for example, by Ginsberg, Med Clin North Am. (2006) 90:1025-36; and West, et al, Annu Rev Sex Res. (2004) 15:40-172).

The phrase “decrease or alleviate symptoms of sexual dysfunction” refers to a decrease in the inhibition of any one or more of the four phases of sexual response (appetite, excitement, orgasm, resolution) described in the DSM-IV. The phrase specifically encompasses increased sexual desire and the enhanced ability to experience orgasm. A particular example of diminished symptoms of sexual dysfunction is an increase in the number, frequency and duration of instances of sexual behavior, including sexual performance, or of subjective sexual arousal.

The phrase “sexual behavior” may or may not involve a partner. Where a partner is involved, sexual behavior comprises arousal, courtship displays and copulation. Arousal (or excitement) consists of a subjective sense of sexual pleasure and accompanying physiological changes. Courtship displays are behaviors intended to or having the effect of arousing a sexual partner and of increasing the arousal of the actor. Copulation may comprise intromission of the penis into the female sexual partner's sexual organs (in heterosexual copulation), orgasm and ejaculation. Where a partner is not involved, sexual behavior may include any combination of touching or erotically manipulating erogenous areas of the genital organs or other erogenous parts of the body (e.g., masturbation); responding to visual stimulation such as pictorial depiction of erotic acts and objects.

The term “sexual arousal” refers to the desire to participate in sexual intercourse and the physical responses that accompany this desire including increased blood flow to genitals and vaginal lubrication.

The term “sexual performance” refers to the act of sexual intercourse.

The phrase “increase sexual arousal and/or performance” refers to an increase in any one or more of the four phases of sexual response (appetite, excitement, orgasm, resolution) described in the DSM-IV. The phrase specifically encompasses increased sexual desire, the enhanced ability to experience orgasm. Increased sexual arousal and/or performance can be measured by the number, frequency and duration of instances of sexual behavior or of subjective sexual arousal.

The term “AMPA receptor” or “AMPA-type glutamate receptor” interchangeably refer to the α-amino-5-hydroxy-3-methyl-4-isoxazole propionic acid (AMPA) receptor, an ionotropic transmembrane receptor for the neurotransmitter glutamate that mediates fast synaptic transmission in the central nervous system (CNS). AMPA receptors are molecules or complexes of molecules present in cells, particularly neurons, usually at their surface membrane, that recognize and bind to glutamate or AMPA. The binding of AMPA or glutamate to an AMPA receptor normally gives rise to a series of molecular events or reactions that result in a biological response. The biological response may be the activation or potentiation of a nervous impulse, changes in cellular secretion or metabolism, or causing cells to undergo differentiation or movement.

The terms “positive modulator of the AMPA receptor” or “upmodulator of the AMPA receptor” interchangeably refer to compounds that bind to the AMPA-type glutamate receptor at a site other than the receptor's active site to increase and/or enhance fast, excitatory transmission.

A “low impact” AMPA modulator refers to a positive AMPA modulator that up-regulates the effect of glutamate, but with reduced risk of inducing seizures in animal models in comparison to other positive AMPA modulators (e.g., “high impact” AMPA modulators).

Categorization of low impact AMPA receptor modulator can be determined by conducting a series of in vitro and in vivo tests using dissociated neurons, hippocampal slices and in vivo physiology, as well as ligand binding experiments. Typically, drugs in the low impact family increase the steady state whole cell patch current in dissociated hippocampal or cortical neurons by no more than 2-fold. Measurements of the fEPSP from the CA1 subregion of hippocampus in the presence of saturating levels of low impact modulator increases the area under the curve (“AUC”) of the response by no more than 50% and usually less than 30%. Furthermore, the increase in the AUC is brought about due to an increase in the amplitude of the response and not to a prolongation of the response caused by slower dissociation of agonist (glutamate). Should a putative modulator fail to give a robust response in these two in vitro assays, the drug is administered to an anesthetized rat that has stimulating and recording electrodes appropriately placed in either the CA1 or dentate gyms in order to measure evoked fEPSP in the respective areas of the hippocampus. Typically, low impact modulators produce a minimum of 10% increase in the fEPSP at appropriate concentrations that vary with the potency of the drug. Finally, a low impact modulator will fail to displace a ligand known to bind to the Aniracetam/cyclothiazide site (the “high impact” site) as characterized by Jin, et al. (J Neurosci. (2005) 25:9027) by X-ray crystallography of the extracellular domains of the GluR2 subunit of the AMPA receptor. Exemplified low impact AMPA upmodulators include CX516 (1-(Quinoxalin-6-ylcarbonyl)piperidine).

A “high impact” AMPA modulator refers to a positive AMPA modulator that increases the effect of glutamate in the brain and up regulates the formation of BDNF (brain-derived neurotrophic factor) much more in comparison to low impact compounds. Categorization of high impact AMPA receptor modulators can be determined by conducting a series of in vitro and in vivo tests using dissociated neurons, hippocampal brain slices and in vivo physiology, as well as ligand binding experiments. Typically, drugs in the high impact family increase the steady state whole cell patch current in dissociated hippocampal or cortical neurons by more than 2-fold. Measurements of the field excitatory postsynaptic potentials (fEPSP) from the CA1 subregion of hippocampus in the presence of saturating levels of a high impact modulator increases the area under the curve (“AUC”) of the response by more than 50%. Furthermore, the increase in the AUC is caused by an increase in the amplitude of the response and not by prolonging the response due to a slower dissociation of agonist (glutamate). Should a putative modulator fail to give a robust response in these two in vitro assays, the drug is administered to an anesthetized rat that has stimulation and recording electrodes appropriately placed in either the CA1 or dentate gyms of hippocampus in order to measure evoked fEPSP. Finally, a high impact modulator will displace a ligand known to bind to the Aniracetam/cyclothiazide site (the “high impact” site), as characterized by Jin, et al. (J Neurosci. (2005) 25:9027), by X-ray crystallography of the extracellular domains of the GluR2 subunit of the AMPA receptor. Exemplified high impact AMPA upmodulators include CX614 (2H,3H,6aH-pyrrolidino[2″,1″-3′,2′]1,3-oxazino[6′,5′-5,4]benzo[e]1,4-dioxan-10-one) and CX689 (2H,7H,8H,5aH-1,3-oxazolidino[2″,3″-3′,2′]1,3-oxazino[6′,5′-5,4]benzo[d]1,3-dioxolan-10-one).

The term “perimenopausal” refers to the period of time around the menopause in which marked menstrual cycle changes occur, often in conjunction with vasomotor symptoms and in which no period of 12 consecutive months of amenorrhea has yet occurred. The median length of the perimenopause is 4 to 5 years (range: 1-9 yrs).

The term “menopause” refers to the period marked by the natural and permanent cessation of menstruation. Menopause usually occurs between the ages of 45 and 55. The date of menopause is established in retrospect, following a full year (i.e., 12 consecutive months) of amenorrhea.

As used herein, “administering” or “administration” refers to oral administration, administration as a suppository, including vaginal or rectal; topical contact, intravenous, intraperitoneal, intramuscular, intralesional, intranasal, intradermal or subcutaneous administration. Administration can be via an implantation of a slow-release device e.g., a mini-osmotic pump, to a subject. Parenteral administration includes any non-oral route, e.g., intravenous, intramuscular, intra-arteriole, intradermal, subcutaneous, intraperitoneal, and intravaginal. Other modes of delivery include, but are not limited to, the use of liposomal formulations, intravenous infusion, transdermal patches, etc. Administration can be local or systemic. In some embodiments, administration is not directly into the brain.

The term “effective amount” refers to a dosage sufficient to produce a desired result. Generally, the desired result is a subjective and/or objective decrease in the symptoms of sexual dysfunction, as measured by the techniques described below.

The term “female mammal” refers to female members of the class of vertebrate that have mammary glands (i.e., mammals) including humans and other primates; domestic mammals including rodents, canines, felines; and agricultural mammals, including equines, ovines, bovines, porcines.

### BRIEF DESCRIPTION OF THE DRAWINGS

FIG. 1 illustrates the Z scores for each female rat given the sexual behavior tests. The means for each behavior and compounds that positively modulate the AMPA receptor were combined for each female to obtain individual Z scores. Animals 1-12 and 15 (mostly on the left of the graph) performed poorly when given vehicle and better when given a positive modulator of the AMPA receptor. Animals 14, 16-18 performed normally when given vehicle and performed worse when given a positive modulator of the AMPA receptor.

FIG. 2 illustrates the group means (±standard error of the mean) for each measure of female sexual behavior for rats when given vehicle and a positive modulator of the AMPA receptor. * p≦0.05.

### DETAILED DESCRIPTION

### 1. Introduction

Surprisingly, it has been found that administering positive modulators of AMPA-type glutamatergic receptors increases sexual arousal, libido and performance activities in female mammals. Although AMPA upmodulators find use in treating sexual dysfunction disorders in male mammals (see, e.g., U.S. Pat. No. 6,083,947), the brain areas and the receptors that mediate male and female sexual arousal are distinct. Furthermore, the present invention is in direct contradiction to the findings of Georgescu and Pfaus, Pharmacol, Biochem Behav (2006) 83:322, who reported that direct infusions of AMPA to the ventromedial hypothalamus (VMH), a brain area that regulates female sexual behavior, decreased both appetitive (arousal) and consummatory (performance) aspects of sexual behavior in female rats.

### 2. Compounds Used to Treat Female Sexual Dysfunction

Compounds useful in the practice of this invention are generally those which amplify (upmodulate) the activity of the natural stimulators of AMPA receptors, particularly by amplifying the excitatory synaptic response. A wide variety of diverse compounds suitable for use in the invention are known in the art. For example, compounds having pharmacophore structures including benzoxazines, benzoyl piperidines, benzoyl pyrrolidines, benzofurazans, benzothiadiazines and biarylpropylsulfonamides find use in the present methods. Such compounds and their synthesis are described for example, in U.S. Pat. Nos. 6,620,808; 6,329,368; 6,274,600; 6,083,947; 6,030,968; 5,985,871; 5,962,447; 5,891,876; 5,852,008; 5,747,492; 5,736,543; 5,650,409 and U.S. Patent Publication No. 2002/0055508, the disclosures of each of which are hereby incorporated herein by reference in their entirety for all purposes. The AMPA upmodulators can be low impact or high impact. Exemplified low impact AMPA upmodulators include CX516 (1-(Quinoxalin-6-ylcarbonyl)piperidine). Exemplified high impact AMPA upmodulators include CX614 (2H,3H,6aH-pyrrolidino[2″,1″-3′,2′]1,3-oxazino[6′,5′-5,4]benzo[e]1,4-dioxan-10-one), and CX689 (2H,7H,8H,5aH-1,3-oxazolidino[2″,3″-3′,2′]1,3-oxazino[6′,5′-5,4]benzo[d]1,3-dioxolan-10-one). Exemplified AMPA upmodulators are taught, for example, in U.S. Pat. Nos. 5,736,543; 5,962,447; 5,985,871; and 6,313,115, and PCT publication WO 03/045315, the disclosures of each of which are hereby incorporated herein by reference.

Additional AMPA receptor potentiators that find use in the present methods include, for example, LY450108 (Czeskis, J Labelled Compounds and Radiopharmaceuticals, (2005) 48:85); N-2-(4-(4-cyanophenol)phenol)propyl-2-propanesulfonamide (LY404187) and (R)-4′-[1-fluoro-1-methyl-2-(propane-2-sulfonylamino)-ethyl]-biphenyl-4-carboxylic acid methylamide (LY503430) (Ryder, et al., J Pharmacol Exp Therapeut (2006) 319:293; LY 392098 (Li, et al., Cell Mol Neurobiol (2003) 23:419); LY451646 (Bai, et al., Neuropharmacol (2003) 44:1013); LY395153 (Linden, et al., Neuropharmacol (2001) 40:1010). AMPA receptor potentiators include sulphonamide derivatives described, for example, in U.S. Pat. Nos. 7,135,487; 6,911,476; 6,900,353; 6,803,484; 6,713,516 and 6,703,425. AMPA receptor potentiators include monofluoroalkyl derivatives described, for example, in U.S. Pat. No. 7,034,045. AMPA receptor potentiators further include other excitatory amino acid receptor modulators described, for example, in U.S. Pat. Nos. 7,125,871 and 7,081,481. The references of this paragraph are hereby incorporated herein by reference in their entirety for all purposes.

Methods for identifying other compounds are routine. They involve a variety of accepted tests to determine whether a given candidate compound is an upmodulator of the AMPA receptor. The primary assay is measurement of enlargement of the excitatory postsynaptic potentials (EPSP) in in vitro brain slices, such as rat hippocampal brain slices.

In experiments of this kind, slices of hippocampus from a mammal such as rat are prepared and maintained in an interface chamber using conventional methods. Field EPSPs are recorded in the stratum radiatum of region CA1b and elicited by single stimulation pulses delivered once per 20 seconds to a bipolar electrode positioned in the Schaffer-commissural projections (see Granger, R. et al., 1993, Synapse, 15:326-329; Staubli, U. et al., 1994a, Proc. Nat. Acad. Sci., 91:777-781; and Staubli, V. et al., 1994b, Proc. Nat. Acad. Sci., 91:11158-11162; Arai, A. et al., 1994, Brain Res., 638:343-346; and Arai, A. et al., 1996, Neuroscience, 75(2):573-85).

The wave form of a normal EPSP is composed of:
an AMPA component, which has a relatively rapid rise time in the depolarizing direction (about 5-10 msec) and which decays within about 20 msec.;
an NMDA component (slow about 30-40 msec rise time and slow about 40-70 msec decay) (the NMDA portion will not appear in normal CSF media, due to the voltage requirement for NMDA receptor channel activation, but in low magnesium media, an NMDA component may appear;
a GABA component in the opposite (hyperpolarizing) direction as the glutamatergic (AMPA and NMDA) components, exhibiting a time course with a rise time of about 10-20 msec and very slow decay (about 50-100 msec or more).

The different components can be separately measured to assay the effect of a putative AMPA receptor enhancing agent. This is accomplished by adding agents that block the unwanted components, so that the detectable responses are essentially only AMPA responses. For example, to measure AMPA responses, an NMDA receptor blocker (e.g., AP-5 or other NMDA blockers known in the art) and/or a GABA blocker (e.g., picrotoxin or other GABA blockers known in the art) are added to the slice. To prevent epileptiform activity in the GABA-blocked slices, known agents such as tetrodotoxin may be used.

AMPA upmodulators useful in the present invention are substances that cause an increased ion flux through the AMPA receptor complex channels in response to glutamatergic stimulation. Increased ion flux is typically measured as one or more of the following non-limiting parameters: at least a 10% increase in decay time, amplitude of the waveform and/or the area under the curve of the waveform and/or a decrease of at least 10% in rise time of the waveform, for example in preparations treated to block NMDA and GABA components. The increase or decrease is preferably at least 25-50%; most preferably it is at least 100%. How the increased ion flux is accomplished (e.g., increased amplitude or increased decay time) is of secondary importance; upmodulation is reflective of increased ion fluxes through the AMPA channels, however achieved.

An additional and more detailed assay is that of excised patches, i.e., membrane patches excised from cultured hippocampal slices; methods are described in Arai, A. et al., 1994, Brain Res., 638:343-346; and Arai, A. et al., 1996, Neuroscience, 75(2):573-85. Outside-out patches are obtained from pyramidal hippocampal neurons and transferred to a recording chamber. Glutamate pulses are applied and data are collected with a patch clamp amplifier and digitized (Arai, A. et al., 1994, Brain Res., 638:343-346; and Arai, A. et al., 1996, Neuroscience, 75(2):573-85).

Because these membrane patches should contain only glutamate receptors, GABAergic currents will not be seen. Any NMDA currents can be blocked as above (e.g., with AP-5).

The central action of a drug can be verified by measurement of field EPSPs in behaving animals (see Staubli, U. et al., 1994a, Proc. Nat. Acad. Sci., 91:777-781) and time course of biodistribution can be ascertained via injection and PET measurement of radiolabeled drug (see Staubli, V. et al., 1994b, Proc. Nat. Acad. Sci., 91:11158-11162).

### Screening of Compounds

A number of compounds belonging to the above-described genus have been shown to up-modulate glutamatergic transmission by augmenting ligand-AMPA receptor complex-activated ion gating. Staubli, U. et al., 1994a, Proc. Nat. Acad. Sci. U.S.A., 9.1:777-781; Staubli, U. et al., 1994b, Proc. Nat. Acad. Sci. U.S.A., 91:11158-11162; Arai, A. et al., 1994, Brain Res., 638:343-346; Granger, R. et al., 1993, Synapse, 15:326-329; all of which are incorporated by reference. These compounds rapidly cross the blood-brain barrier (Staubli, U. et al., 1994b) and increase EPSPs in freely moving rats (Staubli, U. et al., 1994a). Animal experiments indicate that these centrally active modulators improve memory in both rat (Granger, R. et al., 1993; Staubli, U. et al., 1994a) and human models (Lynch et al., 1996, Internat. Clinical Psychopharmacology 11:13; Ingvar et al., submitted to Science, both of which are incorporated by reference).

Once prepared, the compounds of this invention can be screened for their ability to amplify (upmodulate) the activity of the natural stimulators of AMPA receptors, particularly by amplifying excitatory synaptic responses. A variety of accepted tests can be used to determine whether a given compound is an upmodulator of the AMPA receptor. The primary assay is measurement of the enlargement of the EPSP in in vitro brain slices, such as rat hippocampal brain slices.

In experiments of this kind, slices of hippocampus from a mammal, such as rat, are prepared and maintained in an interface chamber using conventional methods. Field EPSPs are recorded in the stratum radiatum of region CA1b and elicited by single stimulation pulses delivered once per 20 seconds to a bipolar electrode positioned in the Schaffer-commissural projections (see, Granger, R. et al., Synapse, 15:326-329 1993; Staubli, U. et al., 1994a, Proc. Nat. Acad. Sci., 91:777-781; and Staubli, V. et al., 1994b, Proc. Nat. Acad. Sci., 91:11158-11162; Arai, A. et al., 1994, Brain Res., 638:343-346; and Arai, A. et al., 1996, Neuroscience, 75(2):573-85). The wave form of a normal EPSP is composed of an AMPA component, which has a relatively rapid rise time in the depolarizing direction (about 5-10 msec) and which decays within about 20 msec.; an NMDA component (slow about 30-40 msec rise time and slow about 40-70 msec decay) (the NMDA portion will not appear in normal or artificial CSF (cerebro-spinal fluid) media, due to the voltage requirement for NMDA receptor channel activation, but in low magnesium media, an NMDA component may appear; a GABA (gamma-aminobutyric acid) component in the opposite (hyperpolarizing) direction as the glutamatergic (AMPA and NMDA) components, exhibiting a time course with a rise time of about 10-20 msec and very slow decay (about 50-100 msec or more).

The different components can be separately measured to assay the effect of a putative AMPA receptor enhancing agent. This is accomplished by adding agents that block the unwanted components, so that the detectable responses are essentially only AMPA responses. For example, to measure AMPA responses, an NMDA receptor blocker (e.g., AP-5 or other NMDA blockers known in the art) and/or a GABA blocker (e.g., picrotoxin or other GABA blockers known in the art) are added to the slice. To prevent epileptiform activity in the GABA-blocked slices, known agents such as tetrodotoxin may be used.

AMPA upmodulators useful in the present invention are substances that cause an increased ion flux through the AMPA receptor complex channels in response to glutamatergic stimulation increased ion flux is typically measured as one or more of the following non-limiting parameters: at least a 10% increase in decay time, amplitude of the waveform and/or the area under the curve of the waveform and/or a decrease of at least 10% in rise time of the waveform, for example in preparations treated to block NMDA and GABA components. The increase or decrease is preferably at least 25-50%; most preferably it is at least 100%. How the increased ion flux is accomplished (e.g., increased amplitude or increased decay time) is of secondary importance; upmodulation is reflective of increased ion fluxes through the AMPA channels, however achieved.

An additional and more detailed assay is that of excised patches, i.e., membrane patches excised from cultured hippocampal slices; methods are described in Arai, A. et al., 1994, Brain Res., 638:343-346; and Arai, A. et al., 1996, Neuroscience, 75(2):573-85.

Outside-out patches are obtained from pyramidal hippocampal neurons and transferred to a recording chamber. Glutamate pulses are applied and data are collected with a patch clamp amplifier and digitized (Arai et al., 1994). Because no GABA is applied to the patch, GABAergic currents will not be elicited. Any NMDA currents can be blocked as above (e.g., with AP-5).

The central action of a drug can be verified by measurement of field EPSPs in behaving animals (see, Staubli et al., 1994a) and time course of biodistribution can be ascertained via injection and subsequent quantitation of drug levels in various tissue samples. Quantitation can be accomplished by methods known to those skilled in the art and will vary depending on the chemical nature of the drug.

### Other Compounds

The above described genus and species of compounds represent merely one example of AMPA upmodulating compounds that may be used to treat sexual dysfunctions according to the present invention. The treatments provided by present invention are not limited to the compounds described above. The present invention also encompasses administering other compounds that enhance the stimulation of alpha-amino-3-hydroxy-5-methyl-isoxazole-4-propionic acid (“AMPA”) receptors in a subject, said enhancement being sufficient to diminish the symptoms of sexual dysfunction. Examples of other such AMPA-selective compounds include 7-chloro-3-methyl-3-4-dihydro-2H-1,2,4 benzothiadiazine S,S, dioxide, as described in Zivkovic et al., 1995, J. Pharmacol. Exp. Therap., 272:300-309; Thompson et al., 1995, Proc. Nat. Acad. Sci. USA, 92:7667-7671.

### 3. Female Mammal Populations Subject to Treatment

The present methods find use in enhancing or increasing sexual arousal and/or performance in any female mammal, including, for example, humans, primates, agricultural animals (e.g., equines, bovine, ovines, porcines, etc.), domestic animals (e.g., canines, felines, etc.) and laboratory animals (e.g., rabbits, rats, mice, hamsters, etc.).

In some embodiments, administration of positive modulators of AMPA-type receptors can be used to enhance or increase libido, sexual arousal and/or performance in female mammals with low estrogen levels in serum. For example, the present methods find use in treating perimenopausal, post-menopausal, ovariectomized, and other female mammals with low estrogen levels. Serum levels of estrogen compounds (e.g., estradiol, estriol, esterone, estrone, etc.) can be measured directly from blood samples using methods well known in the art, for example, by immunoassay. A female mammal with less than about 500 pmol/liter total serum estradiol or less than about 50 ng/ml total serum estrogen is considered to have low estrogen levels. In some embodiments, a female mammal with low estrogen levels will have less than about 400 pmol/l, 350 pmol/l, 300 pmol/l, 250 pmol/l, 200 pmol/l or 150 pmol/l total serum estradiol. A female mammal with less than about 120 pg/ml serum concentration of estradiol is considered to have low estrogen levels. In some embodiments, a female mammal with low estrogen levels will have less than about 100 pg/ml, 80 pg/ml, 60 pg/ml, or 40 pg/ml serum concentration of estradiol. A female mammal with less than about 80 pg/ml serum concentration of estrone is considered to have low estrogen levels. In some embodiments, a female mammal with low estrogen levels will have less than about 70 pg/ml, 60 pg/ml, 50 pg/ml, or 40 pg/ml serum concentration of estrone. See also, for example, Chapter 327 of Harrison's Principles of Internal Medicine, 16th Edition, Kasper, et al., eds, 2005, McGraw-Hill. In some embodiments, measurements can be taken independent of the stage of the menstrual cycle.

Low estrogen levels can also be determined by measuring serum levels of follicle-stimulating hormone (FSH), a hormone that stimulates the ovaries to produce estrogens. A female mammal with less than about 2 IU/liter or more than about 9 IU/liter FSH is considered to have low estrogen levels.

In a related embodiment, positive modulators of AMPA-type receptors can enhance or increase sexual arousal and/or performance in female mammals, particularly humans, that have been diagnosed with a female sexual dysfunction disorder. Female sexual function disorders can be defined through psychological and physical (i.e., medical) analysis.

By psychological diagnosis, female sexual dysfunction is defined by standard approaches used by psychiatrists and clinicians. Diagnoses can be made by consulting the Diagnostic and Statistical Manual of Mental Disorders (DSM IV), 2000, American Psychiatric Association (see, chapter on Sexual and Gender Identity Disorders on pages 535-582, hereby incorporated herein by reference for all purposes). The DSM-IV lists four types of sexual disorders that disturb the process of arousal and the sexual response cycle:
i) Hypoactive sexual desire and sexual aversion disorder: absence of libido and/or aversion to or avoidance or dismissal of sexual prompts or sexual contact;
ii) Female sexual arousal disorder: inability to achieve and progress through the stages of “normal” female arousal;
iii) Female orgasmic disorder is defined as the delay or absence of orgasm after “normal” arousal; and
iv) Sexual Pain Disorders: genital pain before, during, or after intercourse.

Medical conditions also contribute to female sexual dysfunction disorders. When a medical condition contributes to female sexual dysfunction, diagnosis is performed by a specialist clinician. These cases include patients with, for example, inadequate blood flow, nerve-related loss of sensitivity, reduced hormone levels, or disease-related such as diabetes, endocrine disorders of the hypothalamic-pituitary-gonadal axis, and neurological disorders.

### 4. Administration and Formulation

a. Formulation and Routes of Administration

The positive modulators of AMPA-type receptors can be administered to a subject, e.g., a human patient, a domestic animal, including canines and felines, an agricultural animal, including equines, bovines, ovines, porcines, and other mammals, including laboratory mammals (e.g., rabbits, rats, hamster, mice). The AMPA upmodulators can be administered in the form of their pharmaceutically acceptable salts, or in the form of a pharmaceutical composition where the compounds are mixed with suitable carriers or excipient(s) in a therapeutically effective amount, e.g., at doses effective to effect desired increase in sexual arousal and/or performance.

The positive modulators of AMPA-type receptors can be incorporated into a variety of formulations for therapeutic administration. More particularly, AMPA upmodulators of the present invention can be formulated into pharmaceutical compositions by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of one or more AMPA upmodulators can be achieved in various ways, including oral and parenteral, e.g., buccal, intravenous, intravaginal, intradermal, subcutaneous, intramuscular, topical, transdermal, intranasal, etc. administration. Moreover, the compound can be administered in a local or a systemic manner, for example, in a depot or sustained release formulation. In some embodiments, one or more AMPA upmodulators are not administered directly to the brain. In some embodiments, one or more AMPA upmodulators are administered systemically.

Suitable formulations for use in the present invention are found in Remington: The Science and Practice of Pharmacy, 21st Ed., University of the Sciences in Philadelphia (USIP), Lippencott Williams & Wilkins (2005), which is hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In some embodiments, the positive modulators of AMPA-type receptors are prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, hereby incorporated herein by reference). Exemplified extended release formulations that can be used in delivering one or more AMPA upmodulators include, for example, those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, positive modulators of AMPA-type receptors can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, an AMPA upmodulator can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a combination of the invention can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

Systemic administration can also be by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in conventional manner.

In addition to the formulations described previously, the AMPA upmodulators of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Preferred formulations of the compounds are oral preparations, particularly capsules or tablets containing each from about 1 milligram up to about 1000 milligrams of one or more AMPA upmodulators, for example, about 10 mg, 50 mg, 100 mg, 250 mg or 500 mg of AMPA upmodulator.

b. Dosing and Scheduling

Pharmaceutical compositions suitable for use in the present invention include compositions wherein the active ingredients are contained in a therapeutically effective amount. The amount of one or more positive modulators of AMPA-type receptors administered to a subject will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. Generally, an efficacious or effective amount of one or more AMPA upmodulators is determined by first administering a low dose or small amount of the one or more AMPA upmodulators, and then incrementally increasing the administered dose or dosages, until a desired effect of increased sexual arousal and/or sexual performance or decreased symptoms of sexual dysfunction is achieved treated female subject, with minimal or no toxic side effects. Applicable methods for determining an appropriate dose and dosing schedule for administration of a combination of the present invention are described, for example, in Goodman and Gilman's The Pharmacological Basis of Therapeutics, 11th Ed., Brunton, et al., Eds., McGraw-Hill (2006), and in Remington: The Science and Practice of Pharmacy, 2005, supra, both of which are hereby incorporated herein by reference.

Dosage amount and interval can be adjusted individually to provide plasma levels of the active compounds which are sufficient to maintain therapeutic effect. Preferably, therapeutically effective serum levels will be achieved by administering single daily doses, but efficacious multiple daily dose schedules are included in the invention. In some embodiments, the effects of the AMPA up-modulator are realized within about 2-3 days, for example, in under a week. In some embodiments, the AMPA up-modulator can be administered on an “as needed” basis, minutes or hours before engaging in sexual activity. In some embodiments, the AMPA up-modulator is taken chronically, over an extended period of time, for example weeks, months or years. In cases of local administration or selective uptake, the effective local concentration of the drug may not be related to plasma concentration. One having skill in the art will be able to optimize therapeutically effective local dosages without undue experimentation.

Typical dosages for systemic administration range from 1 to 1000 mg/kg, for example, about 20 to 100 mg/kg weight of subject per administration. A typical dosage may be one 10-50 mg tablet taken once or twice a day, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release, as described above.

Dose levels can vary as a function of the specific compound, the severity of the symptoms, and the susceptibility of the subject to side effects. Some of the specific AMPA upmodulators that stimulate glutamatergic receptors are more potent than others. Preferred dosages for a given compound are readily determinable by those of skill in the art by a variety of means. A preferred means is to measure the physiological potency of a given compound that is a candidate for administration, by the method of Davis et al. Psychopharmacology (Berl). 1997 133(2):161-7. Briefly, excised patches and excitatory synaptic responses are measured in the presence of different concentrations of test compounds, and the differences in dosage response potency are recorded and compared. Davis et al. found that one specific compound designated BDP-20 was about ten-fold more potent than another designated BDP-12 in a variety of behavioral (exploratory activity, speed of performance) and physical (excised patches and excitatory synaptic responses) tests. The relative physiological potency was an accurate measure of their behavioral potency. Thus, excised patches and excitatory synaptic responses may be used to gauge the relative physiological (and behavioral) potency of a given compound with regard to a known standard.

Preferred glutamatergic compounds for the treatment of sexual dysfunctions may have a half-life measured from less than 10 minutes to more than 2 hours. In some embodiments, the compound preferably has a rapid onset and short elimination half-life ≦90 min.).

### 5. Assays for Increased Sexual Arousal and/or Performance

Desired effects of increased sexual arousal and/or performance or decreased symptoms of sexual dysfunction can be made by comparing the symptoms and/or behaviors (e.g., sexual activity) in a subject prior to treatment with an AMPA upmodulator with the symptoms and/or behaviors in the same subject after treatment with an AMPA upmodulator. In some embodiments, comparisons are made between a treated and untreated individual.

Tests for assessing increased sexual arousal and/or performance or decreased symptoms of sexual dysfunction can be psychological (objective and subjective) and physical. With respect to subjective psychological evaluation, a female patient can describe subjectively whether treatment with an AMPA upmodulator increased her sexual arousal and/or performance in comparison to her sexual arousal and/or performance before treatment.

With respect to objective self assessment, a female patient can objectively recount the number, frequency and duration of sexual activities or sexual behaviors before and after treatment with one or more positive modulators of an AMPA-type receptor. Psychological evaluations assessing whether treatment with one or more AMPA upmodulators resulted in increased sexual arousal and/or performance or decreased symptoms of sexual dysfunction also can be made by a trained clinician.

a. Diagnostic Criteria for Hypoactive Sexual Desire Disorder (DSM-IV #302.71)

According to the DSM-IV, the essential feature of Hypoactive Sexual Desire Disorder is a deficiency or absence of sexual fantasies and desire for sexual arousal (Criterion A). The disturbance must cause marked distress or interpersonal difficulty (Criterion B). The dysfunction is not better accounted for by another Axis I disorder (e.g., depression, major depression, a psychotic disorder, a cognitive disorder, etc.), and is not due exclusively to the direct physiological effects of a substance (including medications) or a general medical condition (Criterion C). Hypoactive Sexual Desire Disorder can be further divided into subtypes based on onset (lifelong or acquired), context (generalized or situational), and etiological factors (physical, psychological, or a combination thereof).

b. Diagnostic Criteria for Sexual Aversion Disorder (DSM-IV #302.79)

According to the DSM-IV, the essential feature of Sexual Aversion Disorder is the aversion to and active avoidance of genital sexual contact with a sexual partner (Criterion A). The disturbance must cause marked distress or interpersonal difficulty (Criterion B). The dysfunction is not better accounted for by another Axis I disorder (e.g., depression, major depression, a psychotic disorder, a cognitive disorder, etc.) (Criterion C). Sexual Aversion Disorder can be further divided into subtypes based on onset (lifelong or acquired), context (generalized or situational), and etiological factors (physical, psychological, or a combination thereof).

c. Diagnostic Criteria for Female Sexual Arousal Disorder (DSM-IV #307.72)

According to the DSM-IV, the essential feature of Female Sexual Arousal Disorder is a persistent or recurrent inability to attain, or to maintain until completion of the sexual activity, an adequate lubrication-swelling response of sexual excitement (Criterion A). The disturbance must cause marked distress or interpersonal difficulty (Criterion B). The dysfunction is not better accounted for by another Axis I disorder (e.g., depression, major depression, a psychotic disorder, a cognitive disorder, etc.), and is not due exclusively to the direct physiological effects of a substance (including medications) or a general medical condition (Criterion C). Female Sexual Arousal Disorder can be further divided into subtypes based on onset (lifelong or acquired), context (generalized or situational), and etiological factors (physical, psychological, or a combination thereof).

d. Diagnostic Criteria for Female Orgasmic Disorder (DSM-IV #302.73)

According to the DSM-IV, the essential feature of Female Orgasmic Disorder is a persistent or recurrent delay in, or absence of, orgasm following a normal sexual excitement phase (Criterion A). The disturbance must cause marked distress or interpersonal difficulty (Criterion B). The dysfunction is not better accounted for by another Axis I disorder (e.g., depression, major depression, a psychotic disorder, a cognitive disorder, etc.), and is not due exclusively to the direct physiological effects of a substance (including medications) or a general medical condition (Criterion C). Female Orgasmic Disorder can be further divided into subtypes based on onset (lifelong or acquired), context (generalized or situational), and etiological factors (physical, psychological, or a combination thereof).

e. Diagnostic Criteria for Dysparenunia (DSM-IV #302.76)

According to the DSM-IV, the essential feature of Dysparenunia is genital pain that is associated with sexual intercourse (Criterion A). The disturbance must cause marked distress or interpersonal difficulty (Criterion B). The disturbance is not caused exclusively by Vagnismus or lack or lubrication, is not better accounted for by another Axis I disorder (e.g., depression, major depression, a psychotic disorder, a cognitive disorder, etc.), and is not due exclusively to the direct physiological effects of a substance (e.g., a drug of abuse, a medication) or a general medical condition (Criterion C). Dysparenunia can be further divided into subtypes based on onset (lifelong or acquired), context (generalized or situational), and etiological factors (physical, psychological, or a combination thereof).

f. Diagnostic Criteria for Vaginismus (DSM-IV # 306.51)

According to the DSM-IV, the essential feature of Vaginismus is the recurrent or persistent involuntary contraction of the perineal muscles surrounding the outer third of the vagina when vaginal penetration with penis, finger, tampon, or speculum is attempted (Criterion A). The disturbance must cause marked distress or interpersonal difficulty (Criterion B). The dysfunction is not better accounted for by another Axis I disorder (e.g., depression, major depression, a psychotic disorder, a cognitive disorder, etc.), and is not due exclusively to the direct physiological effects of a general medical condition (Criterion C). Vaginismus can be further divided into subtypes based on onset (lifelong or acquired), context (generalized or situational), and etiological factors (physical, psychological, or a combination thereof).

g. Diagnostic Criteria for Sexual Dysfunction Due to a General Medical Condition

According to the DSM-IV, the essential feature of Sexual Dysfunction Due to a General Medical Condition is the presence of clinically significant sexual dysfunction that is judged to be due exclusively to the direct physiological effects of a general medical condition. The sexual dysfunction can involve pain associated with intercourse, hypoactive sexual desire, orgasmic disorder or other forms of sexual dysfunction and must cause marked distress or interpersonal difficulty (Criterion A). There must be evidence from the history, physical examination, or laboratory findings that the dysfunction is fully explained by the direct physiological effects of a general medical condition (Criterion B). The disturbance is not better accounted for by another mental disorder (e.g., Major Depressive Disorder) (Criterion C).

h. Diagnostic Criteria for Sexual Dysfunction Not Otherwise Specified

According to the DSM-IV, sexual dysfunctions that do not meet the criteria of any specific Sexual Dysfunction can be characterized by (1) no (or substantially diminished) subjective erotic feelings despite otherwise normal arousal and orgasm, and/or (2) situations in which the clinician has concluded that a sexual dysfunction is present but is unable to determine whether it is primary, due to a generalized medical condition, or substance induced.

With respect to physical (i.e., medical) evaluation, vaginal blood flow and engorgement can be measured by vaginal photoplethysmography using methods known in the art. See, for example, Marthol and Hilz, Fortschr Neurol Psychiatr. (2004) 72(3):121-35; Rosen, Fertil Steril. (2002) 77 Suppl 4:S89-93; and Laan and Everaerd, Int J Impot Res. (1998) 10 Suppl 2:S107-10. Increased vaginal blood flow and engorgement occurs with sexual arousal. The sensitivity of the clitoris and labia to pressure and temperature can be measured using a biothesiometer according to methods known in the art.

### EXAMPLES

The following examples are offered to illustrate, but not to limit the claimed invention.

### Example 1

### Increasing Female Sexual Arousal and/or Performance Via Systemic Administration of Positive Modulators of AMPA-type Glutamate Receptors

### Material and Methods

All experiments were carried out in accordance with the Institutional Animal Care and Use Committee at the University of California, Irvine, and were consistent with Federal guidelines.

Female Rats. Ovariectomized female Long Evans rats (n=18) were purchased from Charles River Laboratories. Rats were ovariectomized so that their hormonal levels could be controlled since they normally fluctuate during the estrus cycle. First, to give the females sexual experience and to make sure they could still perform sexual behaviors after ovariectomy, females were given full doses of hormone 10 μg estradiol benzoate (EB; Steraloids) 30 hours and 500 μg of progesterone (P) 4 hr before their behavior test. All hormones were dissolved in 0.1 ml safflower oil and injected subcutaneously. To consider a female sexually experienced she needed to receive at least one ejaculation from a male and only these females were used for AMPA upmodulator treatment.

To determine if AMPA upmodulators could increase sexual behavior in these females, they were given lower than normal does of hormone: 2 μg EB 30 hours before experiment and they were not given P.

Behavior Testing. Hormone treated females were given two mating tests per week for four weeks. During each week, half of the rats in each group received either the vehicle or an AMPA upmodulator (CX614 or CX689) for one test, and the opposite treatment for the other in counterbalanced order. To ensure that the tester was blind to the experimental condition of the animal, the vials containing vehicle or AMPA upmodulator were recorded by people other than the tester.

Females were tested under dim light at about the same time each day (e.g., around 11 am-3 pm), which was the beginning of their dark cycle since rats are nocturnal and more active at night. They were given intraperitonial injection of either vehicle or AMPA upmodulator (CX614 or CX689) at 4 mg/kg. The AMPA upmodulators were donated by Cortex Pharmaceuticals (Irvine, Calif.) and dissolved with sonication in 10% of 2-Hydroxypropyl-β-cyclodextrin (FLUKA-Sigma Aldrich, SP) in 0.45% saline, no longer than 30 minutes before use. Ten minutes after the treatment injection, female rats were placed in the testing arena for five minutes to acclimate to their surroundings. Then, a male was introduced and given twenty minutes to mount. The male was changed if it did not mount within ten minutes. The number of proceptive behaviors was recorded which were ear wiggles and hops and darts. The number of times the female performed lordosis, which is the posture assumed by receptive females in response to male mounts, was also recorded as were the number of times females rejected the males. Global statistics were computed and a paired Student's T-test was used for a within animal comparison between tests it received AMPA upmodulator vs. tests it received vehicle.

### Results

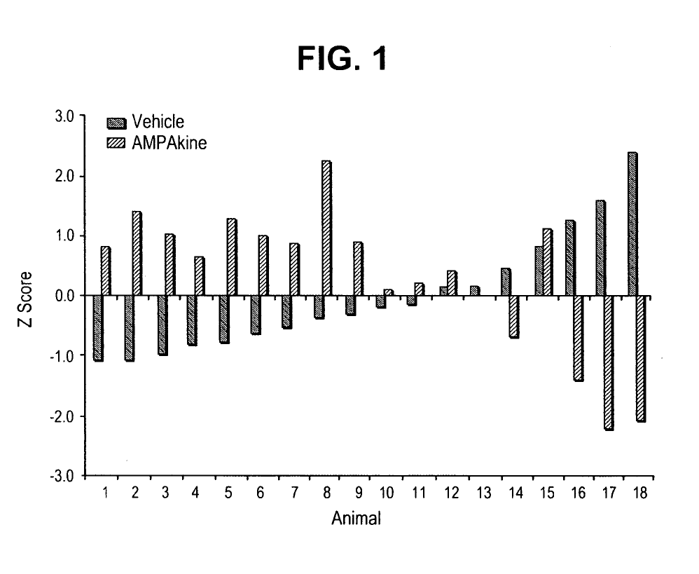

FIG. 1 shows the mean Z scores of the combined measures of all recorded behaviors for each individual female rat given the sexual behavior test. Of the 18 females tested, 13 of them (animals 1-12, 15) performed better on the tests on which they were given an AMPA upmodulator than when they were given vehicle (P<0.05). The females that did not perform poorly when given vehicle (animals 14-18) showed a decline in sexual behavior when given AMPA upmodulator, with the exception of animal 15. So the AMPA upmodulator improved female sexual behavior when the rat was performing poorly but decreased sexual behavior if they performed normally.

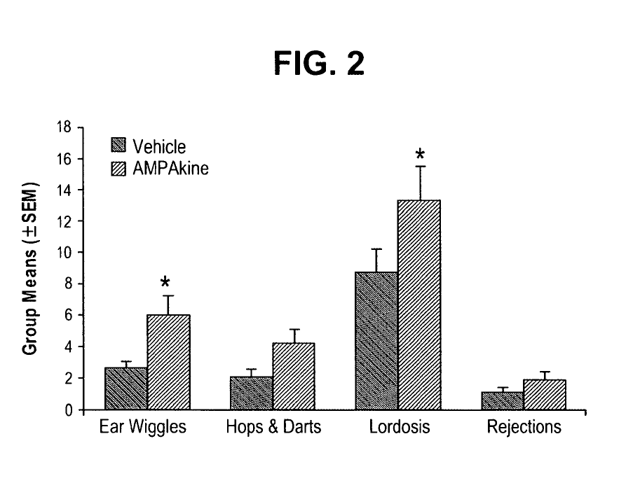

Since female sexual behavior improved with the AMPA upmodulator only if the females were performing poorly when given vehicle, female rats that performed normally (i.e. had means that were one standard deviation above the group mean) were excluded from the analysis of the individual behaviors. FIG. 2 shows the group means for the individual measures of female sexual behavior. The AMPA upmodulators significantly improved receptive or performance aspects of female sexual behavior. Female rats showed more lordosis behavior after administering AMPA upmodulator (13.27±2.22) than vehicle (8.69±1.54; p=0.05). AMPA upmodulators also increased the number of proceptive behaviors performed by the females. Female rats showed significantly more ear wiggles when given AMPA upmodulator (6.0±1.28; mean±SEM) than when administered vehicle (2.67±0.40; p<0.05). Similarly, females performed more hops and darts when given AMPA upmodulator (4.14±0.93) than when given vehicle (2.14±0.42) but this difference only approached significance (p=0.06). The number of rejections was not affected by the AMPA upmodulator treatment.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for decreasing symptoms of sexual dysfunction in a female mammal, said method comprising administering an effective amount of a compound that positively modulates α-amino-3-hydroxy-5-methyl-isoxazole-4-propionic acid (“AMPA”)-type glutamate receptors in said subject, said modulation being sufficient to decrease the symptoms of sexual dysfunction, wherein the female mammal does not otherwise need a positive modulator of the AMPA receptor.

2. The method of claim 1, wherein the female mammal is human.

3. The method of claim 1, with the provision that the mammal not be a rodent.

4. The method of claim 1, wherein decreasing sexual dysfunction comprises increasing sexual arousal.

5. The method of claim 1, wherein decreasing sexual dysfunction comprises increasing sexual performance.

6. The method of claim 1, wherein the female mammal has total serum estradiol levels of less than 500 pmol/l.

7. The method of claim 1, wherein the female mammal has total serum estrogen levels of less than 50 ng/ml.

8. The method of claim 1, wherein the female mammal has a total serum follicle-stimulating hormone levels of less than 2 IU/l or more than 9 IU/l.

9. The method of claim 1, wherein the female mammal is perimenopausal.

10. The method of claim 1, wherein the female mammal is postmenopausal.

11. The method of claim 1, wherein the female mammal is ovariectomized.

12. The method of claim 1, wherein the female mammal has a female sexual dysfunction condition selected from the group consisting of Hypoactive Sexual Desire Disorder (DSM IV #302.71), Sexual Aversion Disorder (DSM-IV #302.79), Female Sexual Arousal Disorder (DSM-IV #302.72), Female Orgasmic Disorder (DSM-IV #302.73), Dyspareunia (DSM IV #302.76) and Vaginismus (DSM-IV #306.51).

13. The method of claim 1, wherein the compound is administered orally.

14. The method of claim 1, wherein the compound is administered parenterally.

15. The method of claim 1, wherein the compound is a low impact positive modulator of the AMPA-type glutamate receptor.

16. The method of claim 1, wherein the compound is a high impact positive modulator of the AMPA-type glutamate receptor.

17. The method of claim 1, wherein the compound is selected from the group consisting of CX516, CX614 and CX689.
